(19) United States
(12) Patent Application Publication
(10) Pub. No.: US 2008/0194958 A1
(43) Pub. Date: Aug. 14, 2008

(54) Method of Compounding and Ultrasound Image

(75) Inventors: Kwang Ju Lee, Seoul (KR); Jong Sik Kim, Seoul (KR); Cheol An Kim, Gyeonggi-do (KR)

Correspondence Address: JONES DAY, 222 EAST 41ST ST, NEW YORK, NY, 10017, US

(73) Assignee: MEDISON, CO., LTD, Hongchun-gun, Kangwon-do (KR)

(21) Appl. No.: 12/063,459

(22) PCT Filed: Dec. 13, 2005
(86) PCT No.: PCT/KR2005/004249

§ 371 Date: Feb. 11, 2008

(30) Foreign Application Priority Data
Aug. 11, 2005 (KR) 10-2005-0073529

(51) Int. Cl. A61B 8/00

(52) U.S. Cl. 600/443

(57) ABSTRACT

In a method of compounding an ultrasound image, ultrasound signals having a transmission frequency are transmitted to a target object at a predetermined steer angle. Then, signals reflected by the target object are received. Based on the received signals, an image frame is formed. By repeating the above steps with different transmission frequencies, two or more image frames are obtained. The obtained image frames are then combined to provide a compound ultrasound image. The steer angle varies based on the transmission frequency.

### TECHNICAL FIELD

The present invention generally relates to a method of compounding an ultrasound image, and more particularly to a method of compounding an ultrasound image through the use of spatial compounding and frequency compounding having a plurality of different transmission frequencies.

### BACKGROUND ART

Ultrasound imaging systems are widely used in the medical diagnostic field since they can obtain an image of a target object through non-invasive means, i.e., by transmitting ultrasound signals to the object and processing their reflection. Conventional three-dimensional (3D) ultrasound imaging systems have an array of ultrasound transducers or probes, which can generate ultrasound pulses and receive echo signals of the ultrasound pulses reflected off an object. In a conventional method, the transducers transmit ultrasound signals of a fixed frequency and then receive signals reflected by a target object. In such a method, only one frequency component corresponding to the fixed frequency is extracted from the received signals to form an ultrasound image.

However, the ultrasound image acquired through such conventional method generally contains speckles, which considerably deteriorate the quality of the ultrasound image. In order to resolve this problem, frequency compounding has been used to reduce such deterioration. The frequency compounding is a method of forming two or more images, which respectively correspond to different transmission frequencies, and combining the formed images in order to provide a desired ultrasound image.

In accordance with a conventional method of frequency compounding, the transducers transmit ultrasound signals of a fixed frequency and receive signals reflected by a target object. This method extracts from the received signals a fundamental frequency component corresponding to the fixed frequency and a second harmonic frequency component. Then, two images are formed respectively based on the fundamental and second harmonic frequency components. The formed images are then combined to provide a compound ultrasound image.

There exists another conventional method, which transmits wide-band signals and receives reflected signals. From the received signals, the method extracts frequency components at a specific interval of frequency. Then, images are formed based on the extracted frequency components and the formed images are combined to provide a compound ultrasound image.

### DISCLOSURE OF INVENTION

### Technical Problem

The conventional methods extract several frequency components from one set of received signals. Therefore, it is required to prepare several circuits in parallel, wherein each of the circuits form an image for the respective frequency component. Further, in the conventional methods, the quality of an ultrasound image is relatively low since the spatial information of the transducers is not considered in combining the images.

### Technical Solution

It is, therefore, an object of the present invention to provide a method of compounding an ultrasound image, which can improve the resolution of the compound ultrasound image by employing modified frequency compounding together with spatial compounding.

In accordance with one aspect of the present invention, there is provided a method of compounding an ultrasound image, comprising the steps of: (a) transmitting ultrasound signals having a predetermined transmission frequency to a target object at a predetermined steer angle; (b) receiving signals reflected by the target object; (c) forming an image frame based on the received signals; (d) repeating steps (a) to (c) with different transmission frequencies to thereby obtain two or more image frames; and (e) combining the obtained image frames to provide a compound ultrasound image, wherein the steer angle varies based on the transmission frequency.

In a more preferred method, the steer angle decreases as the transmission frequency increases, and the steer angle increases as the transmission frequency decreases.

### ADVANTAGEOUS EFFECTS

Since the present invention combines image frames obtained with several different transmission frequencies to provide a compound ultrasound image, it can reduce a speckle noise level and form a smoother ultrasound image.

Further, the ultrasound image, which is compounded in accordance with the present invention, can provide a clearer view of tissue contours and can present even narrow vessels and muscular tissues.

Furthermore, as frequency compounding is used together with spatial compounding, a synergistic effect is provided. Therefore, a high-resolution image can be obtained.

### BRIEF DESCRIPTION OF THE DRAWINGS

The above and other objects and features in accordance with the present invention will become apparent from the following descriptions of preferred embodiments given in conjunction with the accompanying drawings, in which:

FIG. 1 is a functional block diagram of an illustrative ultrasound image display apparatus constructed in accordance with an embodiment of the present invention;

FIG. 2 schematically shows a method of compounding an ultrasound image by frames through the use of frequency compounding in accordance with an embodiment of the present invention; and

FIG. 3 schematically shows a method of compounding an ultrasound image by frames through the use of frequency compounding and spatial compounding in accordance with an embodiment of the present invention.

### BEST MODE FOR CARRYING OUT THE INVENTION

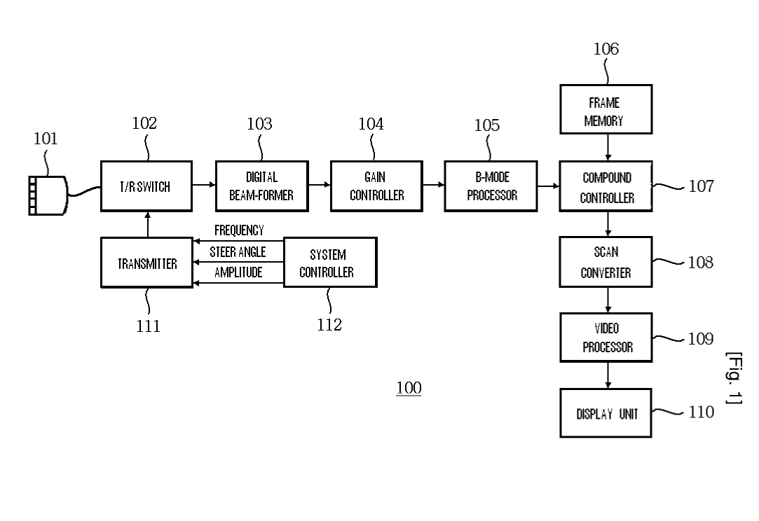

FIG. 1 is a functional block diagram of an illustrative ultrasound image display apparatus constructed in accordance with an embodiment of the present invention.

Referring to FIG. 1, the ultrasound image display apparatus 100 includes: a scan header 101 having a transducer array; a transmit/receive (T/R) switch 102; a transmitter 111; a system controller 112; a digital beam-former 103; a gain controller 104; a brightness-mode (B-mode) processor 105; a frame memory 106; a compound controller 107; a scan converter 108; a video processor 109; and a display unit 110.

The system controller 112 determines the frequency and amplitude of ultrasound signals and a steer angle at which the ultrasound signals are to be transmitted. The transmitter 111 generates ultrasound signals based on the information determined by the system controller 112. The scan header 101 with the transducer array is responsible for transmission of the generated ultrasound signals and reception of signals reflected by a target object (echo signals). The T/R switch 102 serves as a switch that enables the transmission and reception of ultrasound signals to be performed in the same transducer array. As is known in the art, the digital beam-former 103 performs receive-focusing on the echo signals received by the elements in the transducer array. The gain controller 104 performs gain compensation on the receive-focused signals.

The B-mode processor 105 creates a B-mode image frame for a specific frequency component based on the compensated signals. In the B-mode, a magnitude of the echo signal is represented by brightness in an image. To be specific, a bright point represents the presence of a strong reflector in the target object, while a dark point represents the presence of a hypo-echoic portion.

The frame memory 106 may store N number of image frames, which are required to compound an ultrasound image by frames in accordance with the present invention. The compound controller 107 performs spatial compounding with multiple image frames in order to provide a compound ultrasound image. In the context of the invention, the spatial compounding refers to an operation for combining multiple images obtained for several different steer angles to provide a compound ultrasound image.

The scan converter 108 converts the compound B-mode ultrasound image data to a horizontal raster line display format adapted for the display unit 110. The video processor 109 performs image processing on the converted image data in the display format, thereby producing a compound ultrasound image data appropriate for displaying. The display unit 110 displays the compound ultrasound image processed by the video processor 109.

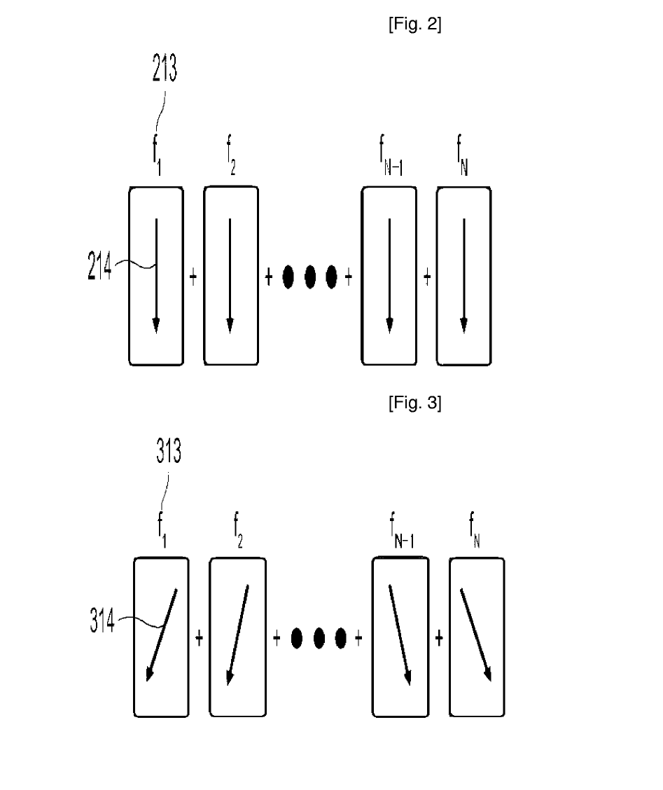

Hereinafter, frequency compounding and spatial compounding in accordance with an embodiment of the invention will be described in more detail with reference to FIGS. 2 and 3.

FIG. 2 schematically shows a method of compounding an ultrasound image by frames through the use of frequency compounding in accordance with an embodiment of the present invention. FIG. 3 schematically shows a method of compounding an ultrasound image by frames through the use of frequency compounding and spatial compounding in accordance with an embodiment of the present invention.

As mentioned above, the compounding of the present invention needs multiple image frames, which are obtained through the use of different transmission frequencies and steer angles. For the formation of each of the frames, the system controller 112 first determines a specific transmission frequency. The transmitter 111 transmits ultrasound signals of the specific transmission frequency. The B-mode processor 105 is used to extract a desired frequency component. The frequency of the desired frequency component varies with frame. The compound controller 107 receives data corresponding to the extracted frequency component from the B-mode processor 105. For the spatial compounding, the system controller 112 controls the transmitter 111 to perform the transmission with a steer angle varying with frame. Therefore, the data received by the compound controller 107 is image data whose frequency component and steer angle vary with frame. The compound controller 107 combines the received image data with compensating positional information based on the steer angle to provide a compound ultrasound image.

Hereinafter, an illustrative method of compounding an ultrasound image, which is in accordance with the present invention, will be described in detail with reference to FIGS. 2 and 3. In such method, a set of ultrasound signals having a frequency are transmitted at a steer angle to a target object and a set of signals reflected by the target object are received. Then, an image frame is formed based on the set of received signals. The method includes the steps of: varying the frequency of the ultrasound signals at every transmission; obtaining at least two frames; combining the obtained frames to provide a compound ultrasound image; and displaying the compound ultrasound image, wherein the steer angle of the ultrasound signals varies based on the frequency thereof.

Hereinafter, there will be described an exemplary method of combining image frames through the use of frequency compounding so as to provide a compound ultrasound image. The frequency compounding is a method of forming two or more image frames respectively corresponding to different transmission frequencies (f₁, f₂ . . . f_{N}) and combining the formed image frames in order to provide a compound ultrasound image. For the formation of each frame, there is determined a transmission frequency 213 (f₁, f₂ . . . f_{N}), a steer angle 214 and an amplitude. The transmission frequency 213 used in this exemplary method may generally be any frequency in a bandwidth supported by a transducer array. For example, if the transducer array has a bandwidth of 2 MHz to 5 MHz, any frequency therein (e.g., 2 MHz, 2.5 MHz, 3 MHz, 3.5 MHz, etc.) may be used.

The attenuation rate of an ultrasound signal depends on its transmission frequency 213. The system controller 112 and the gain controller 104 collaborate for gain compensation of received signals, reflecting differences in attenuation coefficient and steer angle 214 between the transmissions. On the signals received for each transmission frequency, the B-mode processor 105 performs B-mode processing to provide a B-mode image frame. The B-mode image frames compound an ultrasound image, which is displayed through the display unit 110.

In order to use N number of frames to compound an ultrasound image, a time delay for obtaining N−1 number of frames is necessarily incurred in the beginning. That is, a compound ultrasound image cannot be provided until N number of frames are obtained. However, a time delay is not required after accumulating the N−1 number of frames since the transmission frequency preferably varies in rotation. That is, the transmission frequency preferably varies in the order of f₁, f₂ . . . f_{N}, and then f₁ again. For this reason, the present method does not affect the frame rate.

Further, since the transmission frequency (f₁, f₂ . . . f_{N}) varies with frame in the compounding method in accordance with the present invention, it requires only one circuit for the frequency compounding.

The spatial compounding is a method comprising the following steps: transmitting ultrasound signals at several different steer angles 314; obtaining images for the respective steer angles; and combining the obtained images to provide a compound ultrasound image. It is known that the effect of the spatial compounding decreases as the angle between the transmission beams becomes smaller. However, an angle that is too large between them would cause the grating lobe artifact. The maximum angle without causing the grating lobe artifact is represented as follows:

$${θ}_{M} < {sin}^{- 1} ( \frac{λ}{d} - 1 ) \qquad MathFigure 1$$

wherein d represents an element pitch, θ_{M} represents a steer angle of the main lobe, and λ represents a wavelength. The wavelength λ is given as C/f, wherein C is the speed of the ultrasound signal and f is the transmission frequency thereof. Among them, the transmission frequency is adjustable. The Math FIG. 1 clearly shows that the maximum steer angle without causing the grating lobe artifact becomes larger as the transmission frequency becomes smaller.

As stated above, in the present method, the steer angle of the ultrasound signals may preferably vary in association with the frequency. It is further preferable for the compounding to use a high frequency for a small steer angle and a low frequency for a large steer angle. In this way, it becomes possible to maximize the steer angle to thereby acquire an improved effect of compounding without causing the grating lobe artifact.

When frequency compounding is employed to compound an ultrasound image, the contrast resolution is improved due to a speckle reduction effect according to which the size of speckles becomes small and uniform. Further, in case a low frequency component is used, the penetration depth (i.e., the depth up to which an ultrasound image can show) increases. The present invention varies the transmission frequency in a wide frequency range. Accordingly, the present invention naturally employs a transmission frequency, which is lower than the conventional transmission frequency. The lower frequency gives lower attenuation, which increases the penetration depth.

When spatial compounding is employed to compound an ultrasound image, the contrast resolution is improved due to a speckle reduction effect in that the size of speckles becomes small and uniform. Further, due to varying the steer angle, it can provide a better view of a portion shadowed by a bright target. Furthermore, the focusing accuracy becomes regular along the depth.

In the present invention, the frequency compounding is used together with the spatial compounding. In this way, the limitation of the steer angle without the grating lobe artifact can be eased. That is, the steer angle can be higher than the conventional one, thereby improving the effect of the spatial compounding.

### INDUSTRIAL APPLICABILITY

The present invention is applicable to ultrasound imaging systems, which are widely used in the medical diagnostic field for their ability to obtain the image of an object through non-invasive means, i.e., by transmitting ultrasound signals to the object and processing their reflection.

1. A method of compounding an ultrasound image, comprising:
(a) transmitting ultrasound signals having a predetermined transmission frequency to a target object at a predetermined steer angle;
(b) receiving signals reflected by the target object;
(c) forming an image frame based on the received signals;
(d) repeating steps (a) to (c) with different transmission frequencies to thereby obtain two or more image frames; and
(e) combining the obtained image frames to provide a compound ultrasound image,
wherein the steer angle varies based on the transmission frequency.

2. The method of claim 1, wherein the steer angle decreases as the transmission frequency increases, and wherein the steer angle increases as the transmission frequency decreases.

3. A method of compounding an ultrasound image, comprising:
transmitting an ultrasound signal a plurality of times, wherein different frequencies and different steer angles are used for the respective transmissions;
receiving multiple echo signals to form multiple image frames; and
combining the multiple image frames to provide a compound ultrasound image.
